(19) United States
(12) Patent Application Publication
(10) Pub. No.: US 2018/0230416 A1
(43) Pub. Date: Aug. 16, 2018

(54) CELL CHIP AND DYNAMIC DIALYSIS STAINING FOR CELLS

(71) Applicant: National Tsing Hua University, Hsinchu City (TW)

(72) Inventors: Fan-Gang Tseng, Taipei City (TW); Wan-Ying Cho, Hsinchu County (TW)

(73) Assignee: National Tsing Hua University, Hsinchu City (TW)

(21) Appl. No.: 15/584,991

(22) Filed: May 2, 2017

(30) Foreign Application Priority Data
Feb. 13, 2017 (TW) 106104562

(51) Int. Cl. C12M 3/06; B01D 61/00; G01N 33/52

(52) U.S. Cl. CPC C12M 23/16; C12N 2503/00; G01N 33/525; B01D 61/00

(57) ABSTRACT

A cell chip including first and second substrates and a dye dialysis layer disposed therebetween is provided. A cell-assembly region of the dye dialysis layer is disposed corresponding to a first hole of the first substrate, includes second holes and is configured to contain a sample solution containing cells. A size of the second hole is smaller than that of the cell. The cells are arranged on the cell-assembly region in a single layer manner. When a dye enters into the cell-assembly region via the first hole, the dye diffuses from the cell-assembly region to the micro-channel structure due to a concentration difference between the dye and a washing solution in the micro-channel structure. Thereby, the cells are dyed. Also, the washing solution passes in and out the cell chip via the second holes to accelerate the dye diffusion, and a high efficiency dynamic dialysis staining for cells is achieved.

### CROSS-REFERENCE TO RELATED APPLICATION

This application claims the priority benefit of Taiwan application serial no. 106104562, filed on Feb. 13, 2017. The entirety of the above-mentioned patent application is hereby incorporated by reference herein and made a part of this specification.

### BACKGROUND OF THE INVENTION

### Field of the Invention

The invention relates to a chip, and particularly relates to a cell chip.

### Description of Related Art

The observation and cultivation of specific cells are the most basic and most important parts in bio-medical research. The existing way to observe the cells is mostly based on the use of microscopy including the optical microscopy and the fluorescence microscopy. However, a large number of cells in high density are easily stacked together to form multilayered cells. The multilayered cells may cause signal shadowing to generate wrong determination, so as to result in detection errors. Thus, to avoid the detection errors, the cells need to be arranged in a single-layer array.

For instance, in the detection of trace cells, the number of circulating tumor cells (CTCs) is positively correlated with the survival rate and the condition of cancer patients. Thus, the detection and counting of the circulating tumor cells are very important for cancer treatment. However, the currently used detection chip has disadvantages that the cells are liable to stack and the step of cell staining is complicated, which leads to cell loss and death. Thus, it is urgent to develop a cell chip which can simultaneously make the cells be arranged in a single layer manner and improve the efficiency of staining cells in this field.

### SUMMARY OF THE INVENTION

The invention provides a cell chip which can make cells be self-assembled arranged in a single layer manner and be rapidly dyed.

The invention also provides a dynamic dialysis staining for cells to rapidly dye the cells.

The invention provides a cell chip including a first substrate, a second substrate and a dye dialysis layer. The first substrate has at least one first hole. The second substrate has a micro-channel structure. The dye dialysis layer is located between the first substrate and the second substrate and has a cell-assembly region. The cell-assembly region is disposed corresponding to the at least one first hole and separated from the first substrate by a spacing, and the cell-assembly region includes a plurality of second holes. The cell-assembly region is configured to contain a sample solution containing a plurality of cells. A size of each of the second holes is smaller than a particle size of each of the cells. The cells in the sample solution are arranged on the cell-assembly region in a single layer manner, and a liquid portion of the sample solution enters into the micro-channel structure via the second holes. When a dye enters into the cell-assembly region via the first hole, the dye diffuses from the cell-assembly region to the micro-channel structure since there is a concentration difference between the dye and a washing solution flowing in the micro-channel structure. Thereby, the cells are dyed by the dye. The washing solution passes in and out the cell chip via the second holes of the dye dialysis layer to accelerate the diffusion of the dye, and thus a high efficiency dynamic dialysis staining is achieved. Traditionally, cell staining would take 30 minutes in dark environment, and then the waste solution is taken out by a centrifugation machine to finish the entire staining process. However, by using this dynamic dialysis staining method on the cell chip, the traditional staining step and time can be reduced since the staining is accelerated and using centrifugation machine to separate the waste solution and cells is not required.

The invention provides a dynamic dialysis staining for cells including the following steps. The cell chip is provided. A sample solution containing a plurality of cells is dropped into the cell-assembly region of the cell chip via the first hole. A dye is dropped into the cell-assembly region of the cell chip via the first hole to be in contact with the cells. While dropping the dye, a washing solution flows in the micro-channel structure of the cell chip. The dye diffuses from the cell-assembly region to the micro-channel structure since there is a concentration difference between the dye and the washing solution flowing in the micro-channel structure. Thereby, the cells are dyed by the dye. The washing solution passes in and out the cell chip via the second holes of the dye dialysis layer to accelerate the diffusion of the fluorescent dye, and thus a high efficiency dynamic dialysis staining is achieved.

According to an embodiment of the invention, a material of the dye dialysis layer is polydimethylsiloxane (PDMS).

According to an embodiment of the invention, the second holes are arranged in an array.

According to an embodiment of the invention, the spacing between the cell-assembly region and the first substrate is smaller than a particle size of each of the cells.

According to an embodiment of the invention, the micro-channel structure includes a washing solution inlet, a washing solution outlet and a micro-channel located between the washing solution inlet and the washing solution outlet.

According to an embodiment of the invention, the first substrate further includes a washing solution inlet and a washing solution outlet respectively communicated with the washing solution inlet and the washing solution outlet of the micro-channel structure.

According to an embodiment of the invention, the cell chip further includes at least two fixing elements configured to clamp and fix the first substrate, the dye dialysis layer and the second substrate.

According to an embodiment of the invention, the second substrate includes a third substrate and a fourth substrate. The fourth substrate is located between the third substrate and the dye dialysis layer. The third substrate is a light transmissive substrate, and the fourth substrate has a micro-channel opening exposing the third substrate.

According to an embodiment of the invention, the first substrate further includes at least one evaporation hole.

According to an embodiment of the invention, a method that a washing solution flows in the micro-channel structure of the cell chip includes making the washing solution be injected into the micro-channel structure and exhausted from the micro-channel structure continuously by a syringe pump.

According to an embodiment of the invention, after a sample solution containing a plurality of cells is dropped into the cell-assembly region of the cell chip via the first hole, the dynamic dialysis staining for cells further includes sucking a liquid portion of the sample solution via at least one evaporation hole of the first substrate of the cell chip, so that the cells are arranged on the cell-assembly region in a single layer manner by the lateral tensile force with the fluid.

Based on the above, by the combination of the dye dialysis layer with the holes and the micro-channel structure, the cell chip of the invention has both the functions of cell self-assembled arrangement and cell staining. Furthermore, since the cell staining is achieved by diffusion and dynamic dialysis, compared with the principle of density gradient centrifugation with high speed rotation used in the current centrifuges, the invention not only is relatively mild to maintain high viability of the cells, so that the detected cells can be used for subsequent culture, but the cell staining time can be significantly decreased to achieve high efficiency dynamic staining for cells. Additionally, before the cell staining is performed, the cells have been arranged in a single-layer array on the dye dialysis layer. Thus, the phenomenon of multilayered cells can be eliminated, so that the image interpretation is more accurate.

In order to make the aforementioned features and advantages of the disclosure more comprehensible, embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A is a schematic diagram of a cell chip according to an embodiment of the invention, and FIG. 1B is an explosion schematic diagram of the cell chip of FIG. 1A.

FIG. 2A and FIG. 2B are schematic flow diagrams of a use method for a cell chip according to an embodiment of the invention.

### DESCRIPTION OF THE EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

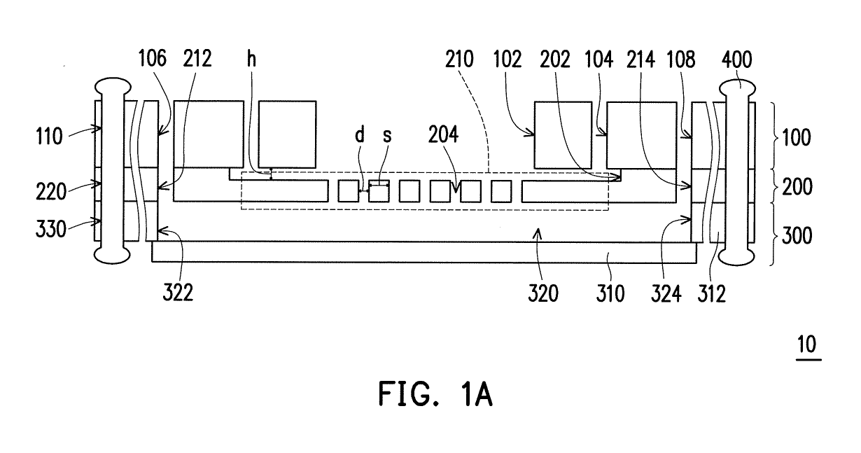

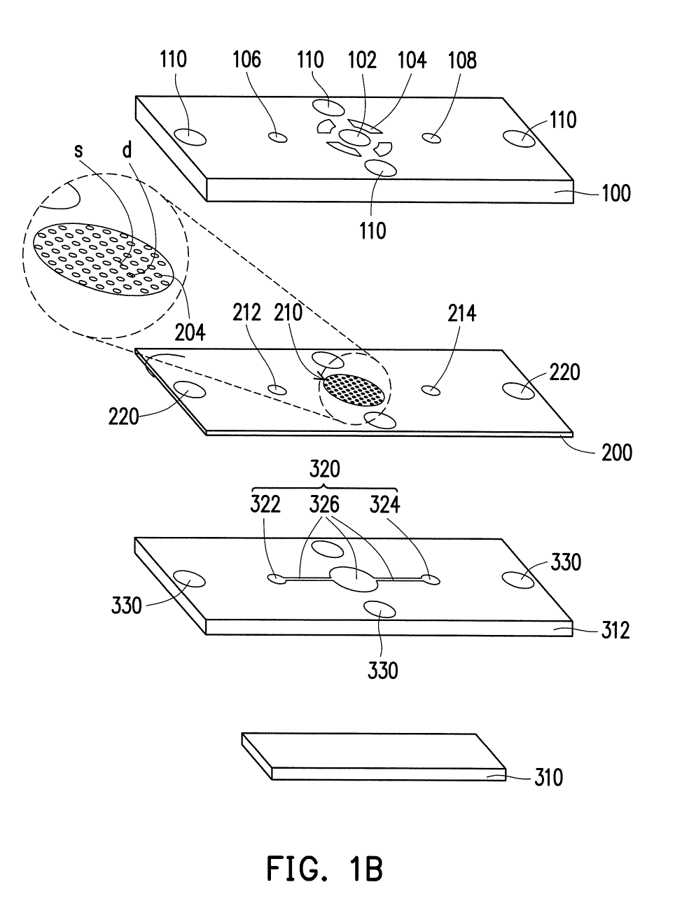

FIG. 1 is a schematic diagram of a cell chip according to an embodiment of the invention, and FIG. 1B is an explosion schematic diagram of the cell chip of FIG. 1A. Referring to FIG. 1A and FIG. 1B at the same time, a cell chip 10 includes a first substrate 100, a dye dialysis layer 200 and a second substrate 300.

The first substrate 100 has at least one first hole 102 configured as an injection port of a sample solution. In the embodiment, the sample solution is a cell suspension containing an appropriate number of cells, for example. The first substrate 100 further includes at least one evaporation hole 104, for example. In the embodiment, the first substrate 100 including a plurality of evaporation holes 104 is used as an example. The evaporation holes 104 have a lenticular shape, for example, and are arranged around the first hole 102 in a circular manner, but the invention is not limited thereto. A size of the first hole 102 is 3 mm to 6 mm, for example. In the embodiment, the first substrate 100 may further include a washing solution inlet 106 and a washing solution outlet 108 located at two opposite sides of the first hole 102, such as located at two opposite sides of the first substrate 100. The washing solution inlet 106 and the washing solution outlet 108 are connected with a syringe pump, for example. In the embodiment, the first substrate 100 may further include at least one pair of fixing element containing holes 110 located at two opposite sides of the first substrate 100 and symmetrically disposed to each other. In the embodiment, the first substrate 100 having four fixing element containing holes 110 is used as an example, but the invention is not limited thereto. A material of the first substrate 100 may be a plastic material, such as polymethylmethacrylate (PMMA). In an embodiment, an upper surface of the first substrate 100 and an inner wall surface of the first hole 102 preferably have an anti-adhesion layer (not shown), such as tridecafluoro-1,1,2,2-tetrahydrooctyl trichlorosilane (FOTS), configured to prevent the overflow of the sample solution or the loss of cells caused by that the cells in the sample solution are adhered onto the first substrate 100. It should be noted that, the invention does not limit the numbers, shapes and configurations of the first hole 102, the evaporation hole 104 and the fixing element containing hole 110.

The dye dialysis layer 200 is located between the first substrate 100 and the second substrate 300. The dye dialysis layer 200 has a cell-assembly region 210. The cell-assembly region 210 is disposed corresponding to the first hole 102 and separated from the first substrate 100 by a spacing h. The spacing h is smaller than a particle size of the cell (or an average particle size of multiple cells), for example. In the embodiment, the spacing h is smaller than or equal to 5 μm, for example. Specifically, the dye dialysis layer 200 has a groove 202. The groove 202 is disposed corresponding to the cell-assembly region 210. The cell-assembly region 210 includes a plurality of second holes 204. The second holes 204 are arranged in an array, for example. In the embodiment, a size d of the second hole 204 is smaller than the particle size of the cell (or the average particle size of multiple cells), such as smaller than or equal to 7 μm. A spacing s between the second holes 204 is larger than the size d of the second hole 204, for example. The spacing s between the second holes 204 is, for example, between 10 μm and 30 μm, such as 20 μm. In the embodiment, the dye dialysis layer 200 having a circular shape is used as an example, but the invention is not limited thereto. In the embodiment, an area of the dye dialysis layer 200 is equal to or larger than an area of the first hole 102, for example. A material of the dye dialysis layer 200 may be a material having a high light transmittance and a high biocompatibility, such as polydimethylsiloxane (PDMS). A thickness of the dye dialysis layer 200 is between 30 μm and 50 μm, such as 40 μm.

In the embodiment, a size of the dye dialysis layer 200 is larger than or equal to that of the first substrate 100, for example. Thus, the dye dialysis layer 200 may further include a washing solution inlet 212, a washing solution outlet 214 and a fixing element containing hole 220 respectively disposed corresponding to the washing solution inlet 106, the washing solution outlet 108 and the fixing element containing hole 110 of the first substrate 100. In another embodiment, when the size of the dye dialysis layer 200 is smaller than that of the first substrate 100, the configurations of the washing solution inlet 212, the washing solution outlet 214 and the fixing element containing hole 220 may be omitted. In other words, the dye dialysis layer 200 may be directly mounted on the second substrate 300 and clamped between the first substrate 100 and the second substrate 300 by clamping the first substrate 100 and the second substrate 300.

In the embodiment, a method of forming the dye dialysis layer 200 includes the following steps, for example. First, a substrate (not shown) is provided, and a photolithography process is performed on the substrate to form a plurality of columns arranged in array as a master mold. The substrate is a silicon wafer, for example. A material of the columns is, for example, a negative photoresist, such as SU-8. Next, a dye dialysis material is injected into a surface of the substrate and filled in gaps between the columns. After the dye dialysis material is cured, the mold is turned over to obtain the dye dialysis layer 200 having a plurality of second holes. Then, the dye dialysis layer 200 is peeled off from the substrate and the master mold. In the embodiment, the dye dialysis material is polydimethylsiloxane, for example. A method of curing the dye dialysis material is a heating method, for example.

The second substrate 300 has a micro-channel structure 320. In the embodiment, the second substrate 300 includes a third substrate 310 and a fourth substrate 312, for example. The fourth substrate 312 is located between the third substrate 310 and the dye dialysis layer 200. The fourth substrate 312 includes a micro-channel opening penetrating the fourth substrate 312, for example, and the third substrate 310 therebeneath is used as a base plate to form a micro-channel structure 320 having a containing space. That is, when the fourth substrate 312 is superimposed on the third substrate 310, the micro-channel opening of the fourth substrate 312 will expose the third substrate 310. The micro-channel structure 320 having the containing space is formed by combining the third substrate 310 with the fourth substrate 312. In the embodiment, the micro-channel structure 320 includes a washing solution inlet 322, a washing solution outlet 324 and a micro-channel 326 located between the washing solution inlet 322 and the washing solution outlet 324. The washing solution inlet 322 and the washing solution outlet 324 are respectively communicated with the washing solution inlet 106 and the washing solution outlet 108, for example. The micro-channel 326 has a region corresponding to the dye dialysis layer 200. Specifically, the region of the micro-channel 326 is larger than or equal to the cell-assembly region 210 and both the two coincide with each other, for example. In the embodiment, the region of the micro-channel 326 is larger than or equal to the first hole 102 in the first substrate 100, for example.

In the embodiment, the fourth substrate 312 further includes at least one pair of fixing element containing holes 330 disposed corresponding to the fixing element containing holes 110 of the first substrate 100, for example. In the embodiment, the fourth substrate 312 having four fixing element containing holes 330 is used as an example, but the invention is not limited thereto. The third substrate 310 is a high light transmissive substrate which facilitates optical observation, such as a glass substrate. A material of the fourth substrate 312 may be a plastic material, such as polymethylmethaciylate. In the embodiment, the third substrate 310 and the fourth substrate 312 are bonded by an adhesion layer, such as an AB gel, for example. In another embodiment, the groove (the groove does not penetrate the substrate) used as the micro-channel structure may be directly formed in the high light transmissive substrate, and thus one of the third substrate and the fourth substrate may be omitted.

In the embodiment, the first substrate 100 and the fourth substrate 312 have an appropriate thickness. The thickness of the first substrate 100 is used as a placement region for the sample solution to provide a sufficient volume for the solution required by the dynamic dialysis diffusion. The fourth substrate is used as a channel for the flow of the dynamic dialysis solution, and the thickness thereof should not be too thick for optical system focal length detection. At the same time, a material of the first substrate 100 and the fourth substrate 312 has a high light transmittance conductive to the observation of optical system, such as an optical microscope or a fluorescence microscope. In the embodiment, the thickness of the first substrate 100 is larger than the thickness of the fourth substrate 312, for example. The thickness of the first substrate 100 is between 4 mm and 6 mm, for example, and the thickness of the fourth substrate 312 is between 1 mm and 4 mm, for example.

In the embodiment, the cell chip 10 further includes at least two fixing elements 400 to clamp and fix the first substrate 100 and the second substrate 300, for example, so that the dye dialysis layer 200 is clamped between the first substrate 100 and the second substrate 300, and the spacing h between the first substrate 100 and the dye dialysis layer 200 is precisely controlled. Therefore, the assembly of the cell chip 10 is completed. The fixing element 400 may be a screw or other elements, but the invention is not limited thereto. After the first substrate 100, the dye dialysis layer 200 and the second substrate 300 are assembled, the dye dialysis layer 200 is suspended above the micro-channel structure 320.

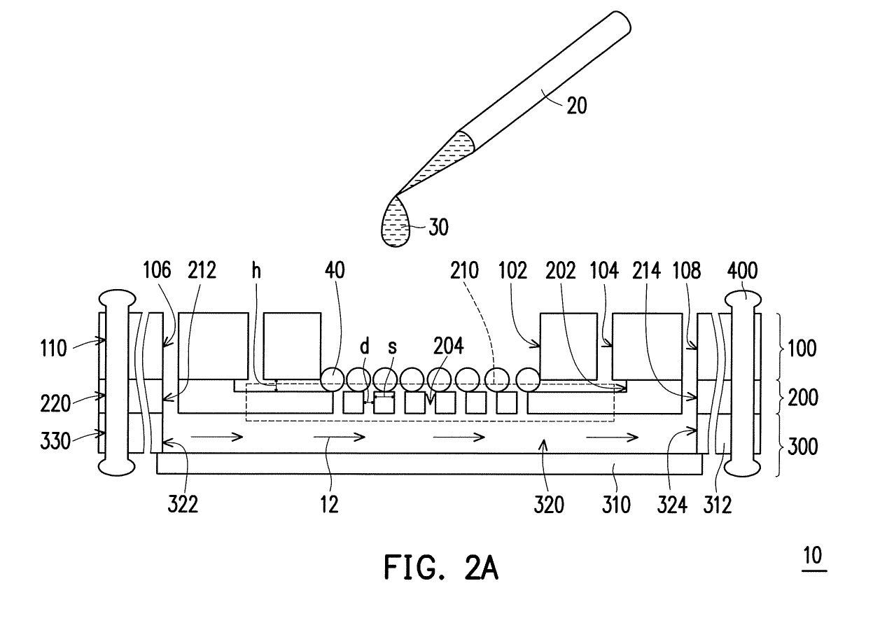

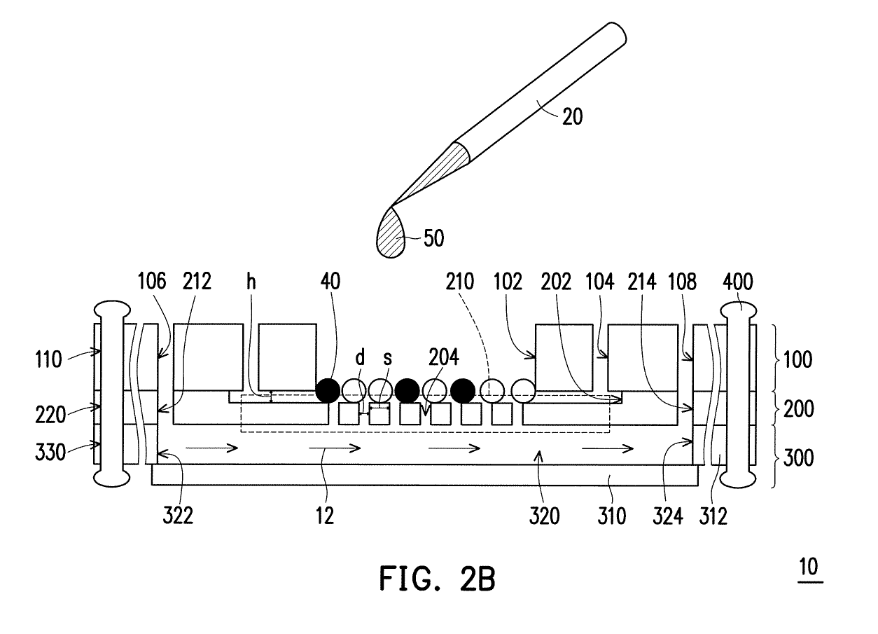

FIG. 2A and FIG. 2B are schematic flow diagrams of a use method for a cell chip according to an embodiment of the invention. Referring to FIG. 2A, first, the cell chip 10 is provided. In the embodiment, after the cell chip 10 is provided, the step of establishing a micro-channel system may be performed. That is, a washing solution 12 is injected into the micro-channel structure 320 of the second substrate 300, so that the washing solution 12 continues to flow in the micro-channel structure 320 of the cell chip 10. The washing solution 12 is a colorless buffer solution, such as phosphate buffered saline (PBS), for example. In the embodiment, the washing solution 12 may be continuously injected into the micro-channel structure 320 via the washing solution inlet 106 and exhausted from the micro-channel structure 320 via the washing solution outlet 108 by a device, such as a syringe pump (not shown).

Next, a sample solution 30 containing a plurality of cells 40 is dropped into the cell-assembly region 210 of the cell chip 10 via the first hole 102. For instance, the sample solution 30, such as a cell suspension, is sampled by a dropper 20 or a pipetman, and the sample solution 30 is added into the cell chip 10 via the first hole 102. The cells 40 of the sample solution 30 are settled down to the cell-assembly region 210 of the dye dialysis layer 200 by the gravity of the solution and the cells 40 themselves. In the embodiment, the cells 40 are arranged onto the cell-assembly region 210 of the dye dialysis layer 200 in a single layer manner by the lateral tensile force of the evaporation of the solution from the evaporation hole 104, for example, and the solution of the suspension enters into the micro-channel structure 320 via the second hole 204. In an embodiment, it further includes directly sucking a liquid portion of the sample solution 30 from the evaporation hole 104 to increase the lateral tensile force, thereby accelerating the cells 40 to be arranged onto the cell-assembly region 210 in a single layer manner. Therefore, the cells 40 in the sample solution 30 are arranged in an array in the cell-assembly region 210 in a self-assembly method to complete the self-assembly with high density cell array. Additionally, it should be mentioned that, the size d of the second hole 204 is designed to be smaller than the particle size of the cells 40, and thus the dye dialysis layer 200 may prevent the cells from flowing out from the second holes 204, so that the loss of the cell number can be avoided, and the liquid such as the liquid portion of the sample solution 30 and the dye diffuses via the channel of the dye dialysis layer.

Referring to FIG. 2B, next, a dye 50 is dropped into the cell-assembly region 210 of the cell chip 10 via the first hole 102 to be in contact with the cells 40. While dropping the dye 50, the washing solution 12 flows in the micro-channel structure 320 of the cell chip 10. For instance, the dye 50, such as an immunofluorescent dye, is sampled by a dropper 20 or a pipetman, for example. The dye 50 is added into the cell chip 10 via the first hole 102, so that the dye 50 flows through the cell-assembly region 210 to be in contact with the cells 40 to dye the cells 40. The dye 50 diffuses from the cell-assembly region 210 to the micro-channel structure 320 due to a concentration difference between the dye 50 and the washing solution 12 flowing in the micro-channel structure 320. Thereby, the cells 40 are dyed by the dye 50. The aforementioned concentration difference means not only the concentration difference between the dye just dropped into the cell-assembly region 210 and the washing solution 12 in the micro-channel structure 320, but also the concentration difference between the dye which has been drooped into the cell-assembly region 210 and the dye which has entered into the micro-channel structure 320 to be diluted by the washing solution 12. Additionally, in the embodiment, the washing solution 12 which enters into the micro-channel structure 320 continuously flows in and out the second holes 204 of the dye dialysis layer 200 to accelerate the diffusion of the dye, so as to achieve dynamic dialysis staining.

The staining speed of the cells can be significantly accelerated, such as the time of traditional staining is shortened, by the diffusion of the dye and the dynamic dialysis staining method of continuously flowing the washing solution in the micro-channel, so as to complete the cell staining with high efficiency. In the embodiment, the washing solution 12 continuously flowing in the micro-channel structure 320 of the cell chip 10 before dropping the dye 50 is used as an example, but the invention is not limited thereto. In other embodiments, the washing solution 12 may continuously flow in the micro-channel structure 320 of the cell chip 10 while or after dropping the dye 50. It should be mentioned that, if the sample solution is to be specific detected or tested, the sample solution needs to be pretreated prior to the use of the cell chip 10, so as to avoid the additional processing process interfering the cell self-assembly array. Furthermore, to avoid foreign substances affecting cell staining, the cell chip 10 may be covered with an upper cover (not shown) thereon to block the first hole 102.

Then, after the cells 40 are dyed for an appropriate period of time, the cell chip 10 is placed under the optical microscope or the fluorescence microscope to be observed. Since the cells 40 are arranged in an array on the cell-assembly region 210 in a single layer manner before dyeing, the phenomenon of multilayered cells can be eliminated under the observation of the microscope, so that the image interpretation is more accurate.

In summary, the cell chip of the invention combines the cell-assembly array chip with the cell staining chip and uses the dye dialysis layer as a platform for carrying the cells and the dye. In the dynamic dialysis staining for cells, since the high efficiency cell staining is achieved by the diffusion and the dynamic dialysis, the disadvantages of the cell loss and cell death in the traditional staining can be reduced, and the method has the advantages of significantly shortening the cell staining time and maintaining cell viability. Additionally, since the cells may be arranged in a single layer manner on the surface of the dye dialysis layer and then dyed, the phenomenon of multilayered cells can be eliminated in the fluorescent image interpretation, so that the image interpretation is more accurate and the detection is more convenient and fast.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A cell chip, comprising:
a first substrate, having at least one first hole;
a second substrate, having a micro-channel structure; and
a dye dialysis layer, located between the first substrate and the second substrate, and having a cell-assembly region, the cell-assembly region being disposed corresponding to the at least one first hole and separated from the first substrate by a spacing, the cell-assembly region comprising a plurality of second holes;
wherein the cell-assembly region is configured to contain a sample solution containing a plurality of cells, a size of each of the second holes is smaller than a size of each of the cells, the cells in the sample solution are arranged on the cell-assembly region in a single layer manner, and a liquid portion of the sample solution enters into the micro-channel structure via the second holes, when a dye enters into the cell-assembly region via the first hole, the dye diffuses from the cell-assembly region to the micro-channel structure since there is a concentration difference between the dye and a washing solution flowing in the micro-channel structure, and thus the cells are dyed by the dye, wherein the washing solution passes in and out the cell chip via the second holes of the dye dialysis layer to accelerate the diffusion of the dye, so as to achieve dynamic dialysis staining.

2. The cell chip according to claim 1, wherein a material of the dye dialysis layer is polydimethylsiloxane (PDMS).

3. The cell chip according to claim 1, wherein the second holes are arranged in an array.

4. The cell chip according to claim 1, wherein the spacing between the cell-assembly region and the first substrate is smaller than a particle size of each of the cells.

5. The cell chip according to claim 1, wherein the micro-channel structure comprises a washing solution inlet, a washing solution outlet and a micro-channel located between the washing solution inlet and the washing solution outlet.

6. The cell chip according to claim 5, wherein the first substrate further comprises a washing solution inlet and a washing solution outlet respectively communicated with the washing solution inlet and the washing solution outlet of the micro-channel structure.

7. The cell chip according to claim 5, wherein the dye dialysis layer further comprises a washing solution inlet and a washing solution outlet respectively communicated with the washing solution inlet and the washing solution outlet of the micro-channel structure.

8. The cell chip according to claim 1, further comprising at least two fixing elements, configured to clamp and fix the first substrate, the dye dialysis layer and the second substrate.

9. The cell chip according to claim 1, wherein the first substrate further comprises at least one evaporation hole.

10. A dynamic dialysis staining for cells, comprising:
providing the cell chip according to claim 1;
dropping a sample solution containing a plurality of cells into the cell-assembly region of the cell chip via the first hole; and
dropping a dye into the cell-assembly region of the cell chip via the first hole to be in contact with the cells, wherein while dropping the dye, a washing solution flows in the micro-channel structure of the cell chip, the dye diffuses from the cell-assembly region to the micro-channel structure since there is a concentration difference between the dye and the washing solution flowing in the micro-channel structure, and thus the cells are dyed by the dye, and the washing solution passes in and out the cell chip via the second holes of the dye dialysis layer to accelerate the diffusion of the dye, so as to achieve dynamic dialysis staining.

11. The dynamic dialysis staining for cells according to claim 10, wherein a method that a washing solution flows in the micro-channel structure of the cell chip comprises making the washing solution be injected into the micro-channel structure and exhausted from the micro-channel structure continuously by a syringe pump.

12. The dynamic dialysis staining for cells according to claim 10, wherein after dropping a sample solution containing a plurality of cells into the cell-assembly region of the cell chip via the first hole, further comprising sucking a liquid portion of the sample solution via at least one evaporation hole of the first substrate of the cell chip, so that the cells are arranged on the cell-assembly region in a single layer manner.
